(19) United States
(12) Patent Application Publication
(10) Pub. No.: US 2009/0214621 A1
(43) Pub. Date: Aug. 27, 2009

(54) Electrostatic Patch For Allergen Screening And Applicator For Same

(75) Inventors: Bertrand Dupont, Aix En Provence (FR); Pierre-Henri Benhamou, Paris (FR)

Correspondence Address: HESLIN ROTHENBERG FARLEY & MESITI PC, 5 COLUMBIA CIRCLE, ALBANY, NY, 12203, US

(73) Assignee: DBV TECHNOLOGIES, Boulogne Billancourt (FR)

(21) Appl. No.: 11/915,926

(22) PCT Filed: May 31, 2005
(86) PCT No.: PCT/FR2005/050397

§ 371 Date: Oct. 17, 2008

(51) Int. Cl. A61K 9/70; A61K 39/35

(52) U.S. Cl. 424/443; 424/275.1

(57) ABSTRACT

A dermal patch comprises a support having electrostatic properties, which is covered with or can be covered with a substance that is intended to be applied to the skin. The surface of the support that is in contact with the substance is provided with microreliefs.

The object of the invention is a skin patch comprising a support provided with electrostatic properties, having an increased adsorption surface.

The object of the invention is also a new type of skin device. More specifically, the object is a device integrating a patch and an applicator, the latter separating from the patch at the instant when said patch, on which is deposited the substance intended to be delivered on the surface of the skin, is applied on said skin.

In the following of the description, the skin patch and the novel type of skin device are more particularly described in connection with screening of the condition of sensitization of a subject to a given food, respiratory, contact allergen, etc., although any other application may be considered.

Two types of tests used for detecting the condition of sensitization towards allergens are marketed today.

First of all, there are tests known under the designation “Finn Chambers®”. This test consists of an adhesive patch on which are stuck small metal aluminium cups at the bottom of which a cellulose disc is simply deposited in the absence of any attachment. In practice, the allergen solution is either prepared extemporaneously or available commercially and deposited on the cellulose disc at the instant of use. The test may therefore contain several cups each filled with different allergens.

This type of test has a certain number of drawbacks, including the essential one of having to be prepared at the moment when it is laid with all the risks related to handling (contamination of a cup by overflow, mistakes, contacts with the adhesive support, random dosage of the active ingredient, etc. . . . ). Further, the absence of firm attachment of the metal support with the cellulose disc which is simply laid down may be at the origin of a movement upon affixing said patch onto the skin. As a result, the glue on the one hand and the cellulose disc on the other hand which probably originally had the required conditions for inertness towards the skin and the allergen, respectively, lose these essential inertness properties when they are used.

A particular application of this type of test, described in document U.S. Pat. No. 4,836,217, is known under the designation of “True Test®”. The originality of the True Test® relatively to Finn Chambers® is the use of a gel directly incorporating the allergen, the gel being directly deposited in the metal cup of the Finn Chambers®. This type of test is reserved for contact allergens mainly of an inorganic nature, which do not a priori pose problems of preservation in a gel unlike organic compounds which risk being modified over time or upon contact with any excipient.

In other words, the first problem which the invention proposes to solve is to develop a skin device:
which is free of any risk of contamination, whether this be when it is manufactured or when it is applied by the final user,
the adhesive portion of which, when it exists, is not touched in any location, so as to provide perfect and long term adhesion of the patch
which allows the active ingredient to be preserved without modifying its allergenic function.

The second problem which the invention proposes to solve is to develop a skin device which may receive in a sufficient amount, both liquid or suspended allergens and allergens in solid form, as dry powder.

Document WO 02/30281 describes a system for applying transdermal patches, i.e., an invasive system. More specifically, the system consists of a push-button, at the end of which the patient positions a transdermal patch i.e. a patch associated with a metal plate provided with spikes with which the skin may be punched, when it is applied. This system, besides the fact that it relates to transcutaneous or transdermal patches, and not to dermal patches, does not solve the problem of the risk of contamination when the user handles the patch before its application.

Document WO 02/089717 describes a dermal patch, the face of which intended to be in contact with the skin is protected by a rigid liner, the liner being firmly attached to two tabs removed at the moment of use. In the same way as previously, the patch and notably its adhesive portion risks being contaminated at the moment of use. Further, it may be expected that a portion of the active substance remains at the surface of the liner once it is removed, thereby modifying the original dosage

Document U.S. Pat. No. 5,236,455 describes a patch placement system for remedying perforations of the eardrum. In practice, the patch is positioned at the end of the distal end of a rod forming a bell, the patch being held in position by producing a vacuum in the cavity formed by the bell. The system therefore requires a complex vacuum pump installation. The actual patch appears as a simple membrane which is dedicated to mechanically blocking the hole and therefore does not contain any active substance.

The applicant succeeded in solving the whole of the problems by reversibly associating a patch to an applicator, the substance intended to be applied on the skin being integral with the patch at the moment of its use.

In other words, the invention relates to a non-invasive one-way-use skin device, which is characterized in that it comprises a patch consisting of a support having at its surface at least one substance intended to be delivered onto the surface of the skin, the back of the patch being reversibly attached onto an applicator, which is detached from the patch after application and adhesion of said patch on the skin.

In other words, by means of this original structure, the final user, at the moment of use, only has to seize the skin device by means of its applicator and deposit the active patch on the skin. Therefore, there is no contact between the active patch and the fingers of the user. Moreover, the proposed system is a non-invasive system in the sense that there is no preparation of the skin by the applicator and/or the patch.

According to a first feature, the skin device combines a patch having a cavity in which the substance is maintained and an applicator having a housing able to receive said cavity. At the cavity, the substance and the patch are therefore not pressing against the skin, so that sweat and air may flow and the maceration effect, capable of causing red spots and irritations may be avoided. Further, blood flow is not altered at the relevant area.

Moreover, in order that the patch may stick to the skin, all or part of the surface of said patch intended to be applied on the skin, is coated with an adhesive material.

According to an essential feature of the invention, mounting of the patch to the surface of the applicator is reversible. Any reversible mounting means may be contemplated.

In a first embodiment, the reversible mount of the patch on the applicator is obtained by means of a so-called “repositionable” adhesive applied on all or part of the back of the patch and/or of the surface of the applicator on which the patch is mounted.

In a second embodiment, the applicator and the patch have cavities of complementary shape, one male cavity (the patch) and the other being a female cavity (the applicator), the reversible mount of the patch on the applicator being obtained by fitting both cavities together. The fit for example results from the dimensions of the female component and from those of the male component.

The reversible mount may also be obtained by means of small teeth or protrusions made around the female portion, said teeth or protrusions will clasp the male portion formed by the patch which is elastic. In both cases, the use of an elastic material for the patch and/or for the applicator promotes fitting and therefore reversible mounting of one on the other.

According to another embodiment, the applicator, a female portion of the assembly, is made in plastic material (polypropylene for example) and includes flexible tabs which are slightly deformed upon mounting the patch and hold it by exerting slight pressure on its periphery.

In another embodiment, the patch, made from a single part in an elastic material, has a shape which facilitates the fitting:
either by deformation,
or by the presence of a groove in which the complementary portion of the applicator will be accommodated.

To avoid any risk of contact between the fingers of the user and the patch, the applicator is provided with means of prehension.

The patch as such, may be of a different nature depending on the substance which it incorporates.

According to a first particularly advantageous embodiment, the substance appears in a dry form, in particular notably as individualised or compressed particles.

In this case, the patch consists of a support provided with electrostatic properties, so that the substance, as individualised or compressed particles, is held in contact with the surface of the support by electrostatic forces.

This type of patch was developed by the Applicant and is specifically described in document WO 02/071950 in connection with “a biologically active substance”, i.e, a substance with a diagnostic, therapeutic, cosmetic or preventative purpose; in the case in point, an allergen. One of the advantages of this patch is that it allows allergens in a pure, native, full or fractionated form to be directly attached thereto, in the absence of any adjuvant.

When the allergen is in liquid form, such as for example milk, the latter is dried or freeze-dried and then milled in order to obtain a powder form.

In another embodiment, the liquid form is not dried but adsorbed on an inert substance. More specifically, in this case, the active substance is deposited as a drop on the electrostatic support on which the dry substance is adsorbed. In this case, the dry substance is an inert substance capable of spreading out the liquid drop containing the biologically active substance on the support on the one hand, and capable of gelling upon contact with this liquid on the other hand. In practice, the inert substance is selected from the group comprising derivatives of dextran or derivatives of celluloses.

Accordingly, the object of the invention is also a skin patch consisting of an electrostatic support intended to be applied on the skin and on which is maintained, by means of electrostatic forces, at least one substance, in the form of as individualised or compressed particles, characterized in that the substance is an inert substance, improving the wettability of the support on the one hand and capable of gelling in contact with a biologically active liquid substance on the other hand, which is in a pure or suspended form.

As already stated, the support is advantageously an electrostatic support.

By the expression “electrostatic support” is meant any support made in a material capable of building up electrostatic charges at its surface and keeping them while developing or holding forces in this way, notably by friction, heating and electrical conditioning, ionization or any other technique known to one skilled in the art.

The electrostatic support or patch then forms what is called an “electret”; it has at its surface a permanent electric field of positive or negative sign, which may be detected by suitable measurement means, known to one skilled in the art.

The charges appearing on either side of the surface of the support may be positive or negative, depending on the material making up said support and on the means used for causing them to appear. In all cases, the positive or negative charges distributed on the surface of the support are at the origin of attractive forces on either conducting materials or not, in the case in point, allergen as individualised or compressed particles. It may also occur that the particles are ionized, which may then cause the same type of electrostatic attractive force between the particles and the support. The electrostatic attraction may also result from polarization of the particles by influence, because of the presence of the electric field.

In practice, any material may be used as a support, provided that it has the required electrostatic properties. In particular, the support is comprised of glass and polymer selected from the group comprising cellulose plastics (CACP), polyvinyl chloride, polypropylenes, polystyrenes polycarbonates, polyacrylics, polyolefins, polyesters, polyethylenes, ethylene vinyl acrylates (EVA).

In the embodiment according to which the electrostatic support has a cavity, the cavity may then be formed directly in the support by hot or cold moulding (thermoforming or plastic injection moulding).

In another embodiment, the cavity is obtained by associating a first electrostatic support corresponding to the bottom of the cavity, and a second support intended to be applied on the skin, the supports being firmly attached at their periphery, by means of a material with small thickness, in practice between 1 and 2 mm, for example in the shape of a ring, if the cavity is circular. In this case, reversible mounting of the patch on its applicator is obtained by a fit.

In an advantageous embodiment, the surface of the electrostatic support is not smooth but has contours. The presence of micro-contours at the surface of the support generates, relatively to a smooth support, the formation of small recesses in which the allergen will be accommodated, so that the available surface area may thereby be increased and therefore the amount of allergen present on the support. The allergen maintained in the recesses is then gradually salted out at the surface of the skin. Of course, the contours are of very small size, of the order of a few micrometers to a few tenths of millimetres in terms of depth.

Accordingly, the invention also relates to a patch, in particular to a skin patch, appearing as a support provided with electrostatic properties, covered or coverable with at least one substance, advantageously a biologically active substance, notably an allergen intended to be applied on the skin, said patch being characterized in that the surface of the support, in contact with the substance, has micro-contours.

The micro-contours may have variable shapes and be obtained in various ways.

In a first embodiment, the contours are obtained from the actual support, for example by hot forming. The contours create recesses and bumps which may have variable shapes, square, hexagonal, circular, radial shapes, etc.

In a second embodiment, the micro-contours obtained by superposition, on the support, of one or more soldered or glued sheets using double-side adhesive film, the support is then called a water-proof and smooth “backing”, the sheets are punched with multiple holes which allow the allergen to be accommodated between the sheets and also in the actual holes.

In a third embodiment, the micro-contours obtained by attaching onto the support, multiple very fine and very short strands or “hairs”, and in a very large amount which forms a sort of carpet capable of receiving and maintaining via capillary action, the substance, notably the deposited allergen. In the case when the support is an electrostatic support, the presence of edges or the small dimensions of the hair, may increase the electrostatic attraction by a tip effect.

The presence of micro-contours allows patches to be prepared for which the support incorporates several biologically active substances. These substances may be introduced into the support at variable depths, so that the first active ingredient is first of all released by sweating before the second active ingredient located in the thickness of the support is released. One of the active ingredients for example plays the role of an adjuvant acting on the properties of the second one (dilution, revelation of its biological action, etc.). It may also act on the skin by promoting transcutaneous transit, by increasing local vascularization and more generally by modifying the local physical or chemical properties.

In the multiple-layered embodiment of the “backing”, with the active ingredient laid out in several layers, it is possible to adjust the release of said active ingredient. With this type of patch, it is thereby possible to control both the released amount and the released flow of the active ingredient.

When the biologically active substance, notably the allergen, then appears as a liquid or a suspension, and it is desired to use it not only as a powder but in its natural form, the allergen is then incorporated into the patch extemporaneously. In this case, the patch should be capable of adsorbing the allergen as a liquid.

In the embodiment described earlier, a dry inert substance, as a powder, is maintained on the support by means of electrostatic forces, the substance being capable of gelling in contact with the active substance as a liquid. In this case, the active substance is put into contact with the inert substance, immediately before applying the patch. The inert substance gels and gradually releases the active substance which it contains.

According to a second embodiment, the support is not an electrostatic support and is in the form of as a woven or non-woven, single-layered or multiple-layered material, based on natural fibers such as cellulose or on synthetic fibers such as polypropylene, alone or as a mixture, the surface weight of the support being 25 and 120 g/m².

In a third embodiment, the support is in the form of a film based on polymers having been subject to physico-chemical treatment which increases the wettability of the adsorbing surface thereby allowing the deposited drop to spread out over the patch.

In a fourth embodiment, the support is a support, advantageously in plastic material, notably as a film, having possibly been subject to physico-chemical treatment improving its wettability, and having contours, the shapes and methods for obtaining them being identical to what was described concerning allergens as a powder.

Unlike dry allergens which may be directly incorporated when the skin device is made, the liquid allergen is adsorbed on the patch when it is used. To do this, the skin device is provided with a lid cooperating with the surface of the applicator so that the patch may be protected during storage. In this case, the lid has an orifice providing passage for the allergen, the lid only being removed once the allergen is adsorbed on the patch.

Another advantage of the lid is to protect the adhesive surface of the patch (when it exists), up to the moment when said patch is laid, to the extent that the latter is not coated with a pealable protective film.

Of course, the patch and the applicator may have variable shapes. Advantageously, the patch and applicator are of a circular shape.

In order to make the skin device free from any possibility or contamination during its storage, the latter is held in a blister which is an enclosure hermetically sealed by means of a pealable cap advantageously.

The invention and the advantages which result therefrom will become better apparent in the following exemplary embodiments, in support of the appended figures.

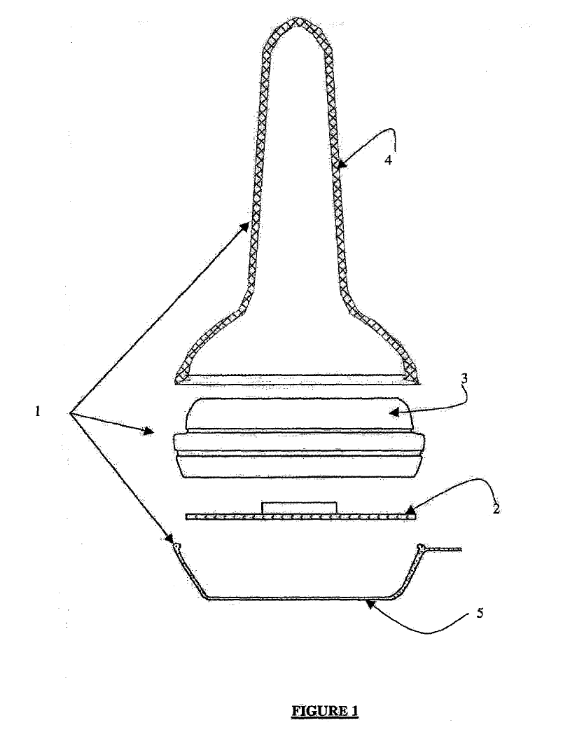

FIG. 1 illustrates an exploded view of the skin device of the invention.

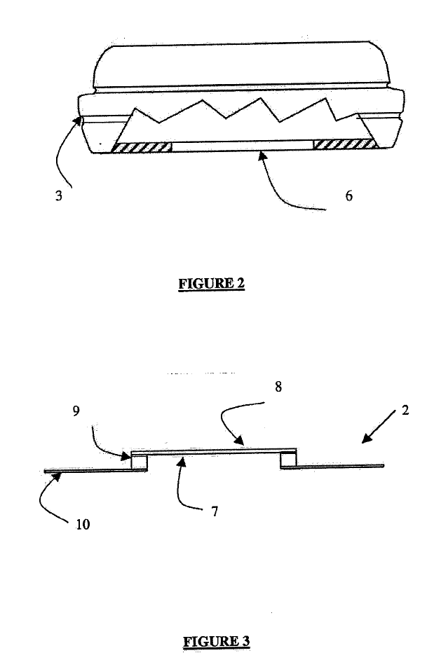

FIG. 2 illustrates the base in a sectional view.

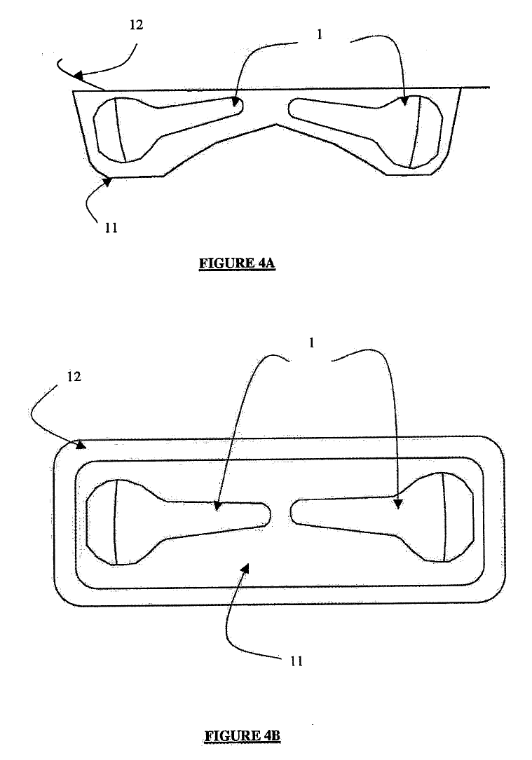

FIG. 3 illustrates a patch capable of incorporating a biologically active substance.

FIG. 4 illustrates an exemplary conditioning of the skin device of the invention.

According to the invention, the skin patch combines an applicator (1) with a patch intended to contain a biologically active substance (2). In the embodiment as illustrated in FIG. 1, the applicator is provided with three parts, a base (3), a means of prehension (4) and a lid (5), respectively, the three parts having a circular section. The means of prehension has a size of about 50 mm so that it may easily be sized by the fingers of the user. In practice, this hollow part is made in plastic. It snaps on to the base (3) with a corresponding shape. More specifically, this base (3) illustrated as a section in FIG. 2, has a housing (6) in which the corresponding cavity (7) of the patch (2) will be accommodated. Finally, once the patch is positioned in its housing, a lid (5) will also be engaged with the base (3) by snapping on the clip so as to protect the patch and possibly the active material which it contains, during storage and at the moment of use. In practice, the diameter of the base is 31 mm.

The patch (2) is more specifically illustrated as a section in FIG. 3. In this particular embodiment, the bottom (8) of the cavity (7) is at least made in an electrostatic material, in the case in point, cellulose acetate. The bottom of the cavity is separated from the patch (2) as such, by means of foam (9) intended to create the cavity (7). The patch (2), as for it, is made in a plastic material covered with an adhesive (10) on all or part of the surface intended to be applied on the skin. To allow reversible mounting of the patch (2) on the surface of the base (3), the diameter of the cavity (7) as well as that of the foam ring (9) is selected so as to fit into the cavity (6).

When the cavity contains a dry allergen as a biologically active substance, this allergen is then incorporated when the skin device is made. On the other hand, when this is a liquid allergen, the latter is incorporated extemporaneously through an orifice not shown provided at the surface of the lid (5).

The invention also relates to devices for storing the skin device illustrated in FIG. 4 in a top view (FIG. 4a) and in a sectional view (FIG. 4b).

More specifically, the storage device appears as a blister (11) containing, in the embodiment as shown, two skin devices and covered with a pealable cap (12). When applying it, the user will therefore have at least one box, or even several boxes assuming that several individualised allergens are tested. Once the blister (11) is opened, the user removes the skin device by seizing it with the prehension means (4). The lid (5) is then removed by unsnapping the clip, and the skin device is then directly applied to the surface of the skin, for example the skin of the baby. The same manoeuvre is repeated for the second skin device and so forth.

When the allergen appears as a liquid, the lid (5) of the skin device has an orifice providing passage of the allergen prepared extemporaneously. The lid is then removed and then the skin device is applied onto the skin of the user.

The advantages of the invention will become well apparent from the foregoing description. The total absence of contamination of the patch by the user as well as its capacity of receiving any type of biological substance, in particular an allergen in dry or liquid form, will be noted in particular.

1. A skin patch comprising a support with electrostatic properties, covered or coverable with at least a substance intended to be applied on the skin, wherein a surface of the support, in contact with the substance, has micro-contours.

2. Patch according to claim 1, wherein the micro-contours are obtained by hot molding the support.

3. Patch according to claim 1, wherein the micro-contours are obtained via superposition, on the support, of one or several soldered or glued layers using double-sided adhesive film, the support being waterproof and smooth, the layers being punched with multiple holes.

4. Patch according to claim 1, wherein the micro-contours are obtained by fixing multiple strands on the support, forming a carpet capable of receiving and maintaining the substance deposed via capillary action.

5. Patch according to claim 1, wherein the support is comprised of glass or polymer chosen from the group consisting of cellulose plastics, polyvinyl chloride polypropylenes, polystyrenes, polyurethanes, polycarbonates, polyacrylics, polyolefines, polyesters, polyethylenes and ethylene vinyl acrylates.

6. Patch according to claim 1, wherein the substance is in the form of individualised or compressed particles.

7. Patch according to claim 6, wherein the substance is a biologically active substance for diagnostic, therapeutic, cosmetic or preventative purposes.

8. Patch according to claim 6, wherein the substance is inert and able to gel upon contact with a liquid biologically-active substance for diagnostic, therapeutic, cosmetic or preventative purposes.

9. A skin patch comprising an electrostatic support intended to be applied on skin and on which is maintained, via electrostatic forces, at least a substance in the form of individualised or compressed particles, wherein the substance is an inert substance improving wettability of the support and able to gel upon contact with a liquid biologically-active substance.

10. Single-use non-invasive skin device, wherein the device comprises a patch comprising a support having on a surface at least a substance destined to be delivered on a surface of the skin, a back of the patch being mounted in a reversible manner on an applicator, the applicator being removed from the patch after applying and sticking the patch onto the skin.

11. Skin device according to claim 10, wherein:
the patch has a cavity in which is maintained the substance;
the applicator has a housing able to receive the cavity.

12. Skin device according to claim 10, wherein all or part of a surface of the patch destined to be applied onto the skin is coated with an adhesive.

13. Skin device according to claim 11, wherein reversible mounting of the patch on the applicator is obtained by adjusting the cavity and the housing.

14. Skin device according to claim 10, wherein the applicator is equipped with a means of prehension.

15. Skin device according to claim 10, wherein the surface of the support has a permanent electrostatic field.

16. Skin device according to claim 10, wherein the substance is in the form of individualised or compressed particles.

17. Skin device according to claim 16, wherein the substance is biologically active for diagnostic, therapeutic, cosmetic or preventative purposes.

18. Skin device according to claim 16, wherein the substance is an inert substance for increasing wettability of the support and able to gel upon contact with a liquid biologically-active substance for diagnostic, therapeutic, cosmetic or preventative purposes.

19. Skin device according to claim 10, wherein the support of the patch is comprised of glass or polymer chosen from the group consisting of cellulose plastics, polyvinyl chloride, polypropylenes, polystyrenes, polyurethanes, polycarbonates, polyacrylics, polyolefines, polyesters, polyethylenes and ethylene vinyl acrylates.

20. Skin device according to claim 10, wherein the support of the patch is of a woven or non-woven type, single or multiple-layered using natural or synthetic fibres, alone or combined with others, the patch weighing between 25˜120 g/m².

21. Skin device according to claim 10, wherein the device further includes a lid cooperating with a surface of the applicator, the lid having an orifice enabling to pass the substance if such substance is a biologically-active substance prepared extemporaneously.

22. Storage device for a skin device having a protective casing in which is stored at least a skin device according to claim 10, the protective casing being hermetically closed.

23. Patch according to claim 7, wherein the substance comprises an allergen.

24. Skin device according to claim 16, wherein the substance comprises an allergen.
